(19) United States
(12) Patent Application Publication
(10) Pub. No.: US 2022/0024176 A1
(43) Pub. Date: Jan. 27, 2022

(54) ANTIMICROBIAL TAPE

(71) Applicant: SENSE-TECH INNOVATION COMPANY, New Taipei City (TW)

(72) Inventors: Meng-Tsung WU, New Taipei City (TW); Kuo-Yuan LING, New Taipei City (TW); Wei-Yao LING, New Taipei City (TW); Chien-Tai FANG, New Taipei City (TW)

(73) Assignee: SENSE-TECH INNOVATION COMPANY, New Taipei City (TW)

(21) Appl. No.: 17/131,656

(22) Filed: Dec. 22, 2020

(51) Int. Cl. B32B 5/16; B32B 27/32; B32B 27/36; B32B 7/12; B32B 7/10; B32B 15/085; B32B 15/09; B32B 15/20

(52) U.S. Cl. CPC B32B 5/16; B32B 27/32; B32B 27/36; B32B 7/12; B32B 7/10; B82Y 5/00; B32B 15/09; B32B 15/20; B32B 2367/00; B32B 2323/10; B32B 2307/518; B32B 15/085

(57) ABSTRACT

The present disclosure discloses an antimicrobial tape comprising a tape layer, a connection layer, and an antimicrobial layer. The connection layer is disposed on the tape layer, and the connection layer consists of angstrom-level silicon dioxide. The antimicrobial layer is disposed on the connection layer. The tape layer and the antimicrobial layer are connected by the connection layer consisting of angstrom-level silicon dioxide to increase the connection strength between the tape layer and the antimicrobial layer. The tape layer may be used to cover a surface of an object, and the antimicrobial layer is located on an outer surface. Therefore, after a user touching and using the object, the object has been sterilized through the antimicrobial layer of the antimicrobial tape.

### CROSS REFERENCE TO RELATED APPLICATION

This application claims the priority benefit of Taiwan Patent Application Number 109209532, filed on Jul. 24, 2020, the full disclosure of which is incorporated herein by reference.

### BACKGROUND

### Technical Field

The present disclosure relates to the technical field of a plastic surface, particularly to an antimicrobial tape.

### Related Art

At present, many infectious diseases caused by viruses or bacteria have appeared in the world, which seriously affect human health and cause large-scale damage to the human living environment. Furthermore, bacteria or viruses sometimes mutate due to environmental factors and develop drug resistance, etc. How to effectively prevent and reduce the damage of viruses and bacteria to the human living environment is one of the issues that humans need to pay attention to. Antimicrobial materials have also become a goal and direction of research and development by researchers.

### SUMMARY

The embodiments of the present disclosure disclose an antimicrobial tape.

The present disclosure discloses an antimicrobial tape comprising a tape layer, a connection layer, and an antimicrobial layer. The connection layer is disposed on the tape layer, and the connection layer consists of angstrom-level silicon dioxide. The antimicrobial layer is disposed on the connection layer.

In the embodiments of the present disclosure, the antimicrobial tape has a tape layer, a connection layer, and an antimicrobial layer. The antimicrobial layer may provide an antimicrobial effect. The tape layer and the antimicrobial layer is connected through the connection layer to increase the connection strength between the tape layer and the antimicrobial layer.

It should be understood, however, that this summary may not contain all aspects and embodiments of the present disclosure, that this summary is not meant to be limiting or restrictive in any manner, and that the disclosure as disclosed herein will be understood by one of ordinary skill in the art to encompass obvious improvements and modifications thereto.

### BRIEF DESCRIPTION OF THE DRAWINGS

The features of the exemplary embodiments believed to be novel and the elements and/or the steps characteristic of the exemplary embodiments are set forth with particularity in the appended claims. The Figures are for illustration purposes only and are not drawn to scale. The exemplary embodiments, both as to organization and method of operation, may best be understood by reference to the detailed description which follows taken in conjunction with the accompanying drawings in which:

FIG. 1 is a schematic diagram of the antimicrobial tape of the first embodiment of the present disclosure;

FIG. 2 is a schematic diagram of the antimicrobial tape of the second embodiment of the present disclosure;

FIG. 3 is a schematic diagram of the antimicrobial tape of the third embodiment of the present disclosure;

FIG. 4 is a schematic diagram of the antimicrobial tape of the fourth embodiment of the present disclosure; and

FIG. 5 is a schematic diagram of the antimicrobial tape of the fifth embodiment of the present disclosure.

### DETAILED DESCRIPTION OF THE EMBODIMENTS

The present disclosure will now be described more fully hereinafter with reference to the accompanying drawings, in which exemplary embodiments of the disclosure are shown. This present disclosure may, however, be embodied in many different forms and should not be construed as limited to the embodiments set forth herein. Rather, these embodiments are provided so that this present disclosure will be thorough and complete, and will fully convey the scope of the present disclosure to those skilled in the art.

Certain terms are used throughout the description and following claims to refer to particular components. As one skilled in the art will appreciate, manufacturers may refer to a component by different names. This document does not intend to distinguish between components that differ in name but function. In the following description and the claims, the terms “include/comprising” and “comprise/comprising” are used in an open-ended fashion, and thus should be interpreted as “comprising but not limited to”. “Substantial/substantially” means, within an acceptable error range, the person skilled in the art may solve the technical problem in a certain error range to achieve the basic technical effect.

The following description is of the best-contemplated mode of carrying out the disclosure. This description is made for the purpose of illustration of the general principles of the disclosure and should not be taken in a limiting sense. The scope of the disclosure is best determined by reference to the appended claims.

Moreover, the terms “include”, “contain”, and any variation thereof are intended to cover a non-exclusive inclusion. Therefore, a process, method, object, or device that includes a series of elements not only includes these elements, but also includes other elements not specified expressly, or may include inherent elements of the process, method, object, or device. If no more limitations are made, an element limited by “include a/an . . . ” does not exclude other same elements existing in the process, the method, the article, or the device which includes the element.

In the following embodiment, the same reference numerals are used to refer to the same or similar elements throughout the disclosure.

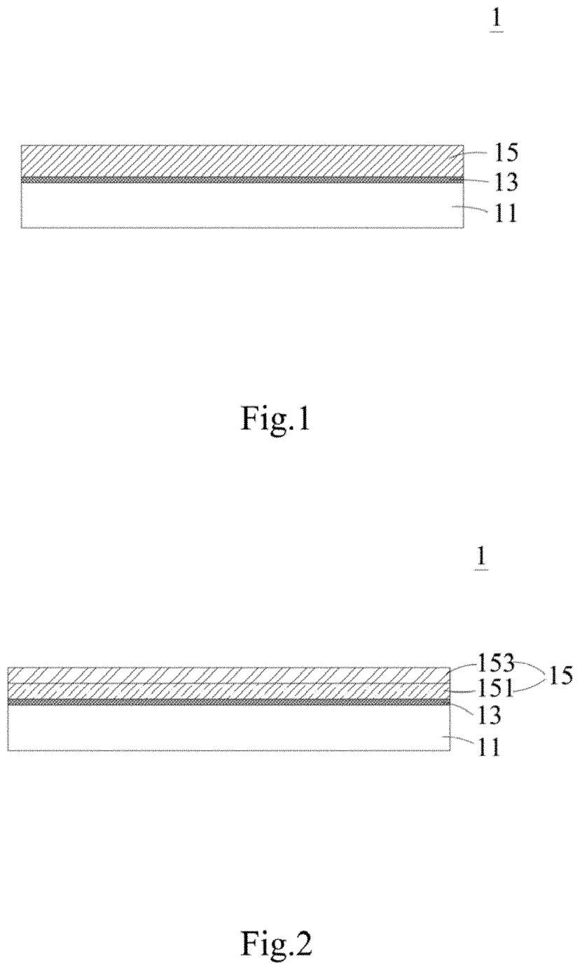

Refer to FIG. 1, which is a schematic diagram of the antimicrobial tape of the first embodiment of the present disclosure. As shown in the figure, the antimicrobial tape 1 of the present embodiment comprises a tape layer 11, a connection layer 13, and an antimicrobial layer 15. The connection layer 13 is disposed on the tape layer 11, and the connection layer 13 consists of angstroms-level silicon dioxide. The antimicrobial layer 15 is disposed on the connection layer 13. The material of the tape layer 11 is biaxially oriented polypropylene film (BOPP) or resin(PET).

In the present embodiment, the connection layer 13 is disposed between the tape layer 11 and the antimicrobial layer 15. The structural shape of the connection layer 13 consisting of angstrom-level silicon dioxide is a half-sheet shape. Therefore, structural pores of the connection layer 13 are increased. When the tape layer 11 and the antimicrobial layer 15 are respectively disposed on opposite surfaces of the connection layer 13, the tape layer 11 and the antimicrobial layer 15 respectively attach to and fill in the structural pores on the opposite surfaces of the connection layer 13. The connection strength of the tape layer 11 and the antimicrobial layer 15 attached to connection layer 13 is increased. Therefore, shedding of antimicrobial material of the antimicrobial layer 15 is reduced and service life of the antimicrobial tape 1 is increased.

Refer to FIG. 2, which is a schematic diagram of the antimicrobial tape of the second embodiment of the present disclosure. As shown in the figure, the difference between the present embodiment and the first embodiment is the antimicrobial layer 15. In the present embodiment, the antimicrobial layer 15 further comprises a base layer 151 and a surface antimicrobial layer 153. The surface antimicrobial layer 153 is disposed on the base layer 151, and the base layer 151 is disposed on the connection layer 13. The base layer 151 comprises chromium or aluminum oxide. The surface antimicrobial layer 153 comprises silver, titanium oxide, zinc oxide, or magnesium oxide. A particle diameter of the material of the surface antimicrobial layer 153 is less than 50 nanometers, wherein the particle diameter is preferably less than 30 nanometers. The surface antimicrobial layer 153 described above is disposed on the base layer 151, therefore the connection strength between the base layer 151 and the surface antimicrobial layer 153 is furtherly enhanced.

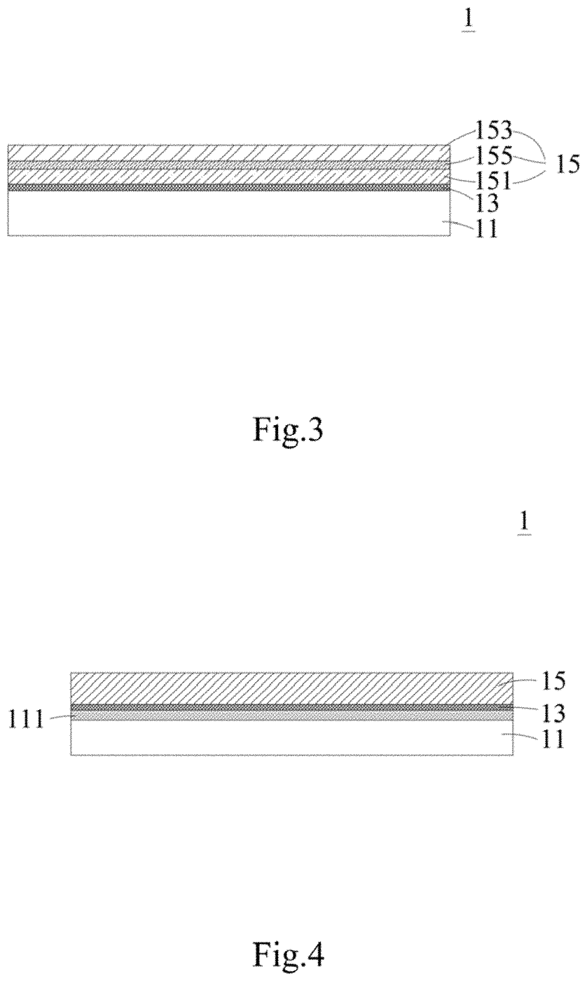

Refer to FIG. 3, which is a schematic diagram of the antimicrobial tape of the third embodiment of the present disclosure. As shown in the figure, in the present embodiment, the difference between the present embodiment and the second embodiment is the antimicrobial layer 15. In the present embodiment, the antimicrobial layer 15 further comprises a connection antimicrobial layer 155. The connection antimicrobial layer 155 is disposed between the base layer 151 and the surface antimicrobial layer 153, and the connection antimicrobial layer 155 consists of angstrom-level silicon dioxide. The connection antimicrobial layer 155 and the connection layer 13 of the present embodiment have the same structural composition and effect. Therefore, the description thereof may be omitted.

Refer to FIG. 4, which is a schematic diagram of the antimicrobial tape of the fourth embodiment of the present disclosure. As shown in the figure, the difference between the present embodiment and the first embodiment is the tape layer 11. In the present embodiment, the tape layer 11 further comprises a rough surface 111. The rough surface 111 is located on an upper surface of the tape layer 11. The rough surface 111 has a plurality of nano-level pores. The rough surface 111 is formed through ion bombardment from a surface of the tape layer 11. When the connection layer 13 is disposed on the rough surface 111 of the tape layer 11, the connection layer 13 attach to and fill in a plurality of nano-level pores. Therefore, the connection strength between the connection layer 13 and the tape layer 11 is increased.

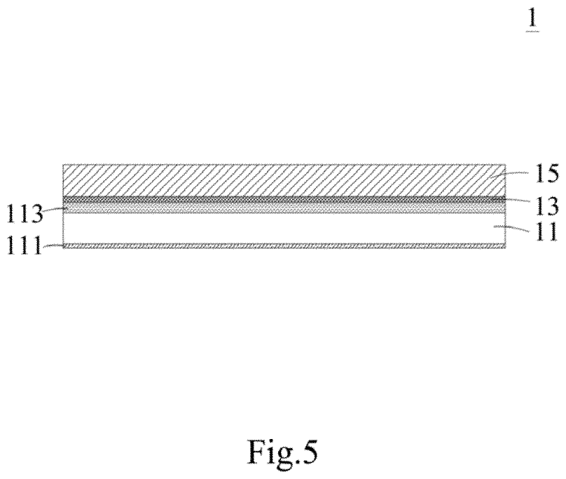

Refer to FIG. 5, which is a schematic diagram of the antimicrobial tape of the fifth embodiment of the present disclosure. As shown in the figure, the difference between the present embodiment and the fourth embodiment is the tape 11. In the present embodiment, the tape layer 11 further comprises an adhesive surface 113, and the adhesive surface 113 is located on a lower surface of the tape layer 11. A user can use the adhesive surface 113 of the tape layer 11 to attach and cover an object. An outer surface of the object is covered by the antimicrobial layer 15. Therefore, when the user touches and uses the object, the object covered by the antimicrobial layer 15 has an antimicrobial effect.

In summary, the present disclosure provides an antimicrobial tape comprising a tape layer, a connection layer, and an antimicrobial layer. The antimicrobial layer may provide antimicrobial effects, and the tape layer and the antimicrobial layer are connected through the connection layer. Therefore, the connection strength between the tape layer and the antimicrobial layer is increased, and the service life of the antimicrobial layer is increased.

Although the present disclosure has been explained in relation to its preferred embodiment, it does not intend to limit the present disclosure. It will be apparent to those skilled in the art having regard to this present disclosure that other modifications of the exemplary embodiments beyond those embodiments specifically described here may be made without departing from the spirit of the disclosure. Accordingly, such modifications are considered within the scope of the disclosure as limited solely by the appended claims.

What is claimed is:

1. An antimicrobial tape, comprising:
a tape layer;
a connection layer disposed on the tape layer, wherein the connection layer consists of angstrom-level silicon dioxide; and
an antimicrobial layer disposed on the connection layer.

2. The antimicrobial tape according to claim 1, wherein the antimicrobial layer comprises a base layer and a surface antimicrobial layer, the surface antimicrobial layer is disposed on the base layer, and the base layer is disposed on the connection layer.

3. The antimicrobial tape according to claim 2, wherein the surface antimicrobial layer comprises silver, titanium oxide, zinc oxide, or magnesium oxide.

4. The antimicrobial tape according to claim 3, wherein a particle diameter of a material of the surface antimicrobial layer is less than 50 nanometers.

5. The antimicrobial tape according to claim 4, wherein the particle diameter of the material of the surface antimicrobial layer is preferably less than 30 nanometers.

6. The antimicrobial tape according to claim 2, wherein the base layer comprises chromium or aluminum oxide.

7. The antimicrobial tape according to claim 2, wherein the antimicrobial layer further comprises a connection antimicrobial layer, the connection antimicrobial layer is disposed between the base layer and the surface antimicrobial layer, and the connection antimicrobial layer is consisting of angstrom-level silicon dioxide.

8. The antimicrobial tape according to claim 1, wherein the tape layer comprises a rough surface, the connection layer disposed on the rough surface.

9. The antimicrobial tape according to claim 8, wherein the rough surface has a plurality of nano-level pores.

10. The antimicrobial tape according to claim 1, wherein the tape layer further comprises an adhesive surface.

11. The antimicrobial tape according to claim 1, wherein a material of the tape layer is biaxially oriented polypropylene film (BOPP) or resin (PET).
